(19) United States
(12) Patent Application Publication
(10) Pub. No.: US 2025/0369041 A1
(43) Pub. Date: Dec. 4, 2025

(54) 3-DIMENSIONAL ROBOTIC SEQUENCING DEVICE

(71) Applicant: ESBIOLAB, LLC, San Diego, CA (US)

(72) Inventors: Liangliang Qiang, San Diego, CA (US); Wei Li, San Diego, CA (US)

(21) Appl. No.: 19/124,924

(22) PCT Filed: Oct. 26, 2023
(86) PCT No.: PCT/US2023/035993

§ 371 Date: Apr. 28, 2025

Related U.S. Application Data
(60) Provisional application No. 63/381,070, filed on Oct. 26, 2022.

(51) Int. Cl. C12Q 1/6869; B01L 3/00

(52) U.S. Cl. CPC C12Q 1/6869; B01L 3/502715; B01L 2200/027; B01L 2200/16; B01L 2300/0819; B01L 2300/0867

(57) ABSTRACT

A nucleotide sequencing device includes a plurality microfluidic chips configured for nucleic acid sequencing and a reagent dispensing manifold with at least one dispensing port wherein the manifold is operable to move in at least two dimensions relative to the plurality of microfluidic chips to allow the reagents from the manifold to be dispensed to the microfluidic chips without the use of a common line between the dispensing port and microfluidic chips when certain reagents are requested.

### RELATED APPLICATION

This application claims priority from U.S. Provisional Application No. 63/381,070, filed Oct. 26, 2022, the subject matter of which is incorporated herein by reference in its entirety.

### BACKGROUND

Microfluidic devices/chips are widely used in biological/medical applications. Particularly in next-generation sequencing (NGS) systems, such devices are used to immobilize template nucleic acid molecules derived from biological samples and then introduce a repetitive flow of sequencing-by-synthesis reagents to attach labeled nucleotides to specific positions in the template sequences. A series of label signals are detected and decoded to reveal the nucleotide sequences of the template molecules, e.g., immobilized and/or amplified nucleic acid template molecules attached to an internal surface of microchannels of the device.

Typical devices use integrated pumps and valves to control the flow/delivery of different reagents. A common line between valve and microfluidic chips is unavoidable and extra wash volume is required during reagent exchange. Large amounts of reagents are wasted to flush the common line especially when microfluidic channels are very small.

### SUMMARY

This application describes new designs of a nucleotide sequencing device that saves reagents during fluidic exchange. The nucleotide sequencing device includes a plurality of microfluidic chips configured for nucleotide sequencing and a reagent dispensing manifold that is not continuously connected to the microfluidic chips for dispensing or delivering nucleotide sequencing reagents to each microfluidic chip. Each microfluidic chip includes a microchannel having an inlet configured to receive a reagent and an outlet in fluid communication with a waste collection unit. The reagent dispensing manifold includes at least one reagent dispensing port. The reagent dispensing manifold is operable to move in at least two dimensions or three dimensions relative to the plurality of microfluidic chips when certain reagents are requested by chips to allow the at least one dispensing port to form a leak-proof fluid connection with each inlet of the microchannels after movement. The at least one dispensing port is configured to be disconnected from the inlet of the microchannel after delivery of the reagent and/or upon movement of the reagent dispensing manifold. Advantageously, in contrast to conventional nucleotide sequencing devices, a common line between the valve/pump and microfluidic channels is not shared. Not sharing a common line for delivering a reagent to the microchannels can significantly improve the usage of expensive reagents and reduce the cost of operation of the nucleotide sequencing device.

In some embodiments, the device includes a platform that extends generally in an x-y plane of an x-y-z coordinate system. The plurality of microfluidic chips are arranged on a surface of the platform and the reagent dispensing manifold is positioned over the platform in a z direction. The device also includes a robotic arm that can move the reagent dispensing manifold in an x, y, and/or z direction to position the reagent dispensing port over the inlet of a respective microfluidic chip.

In some embodiments, the device includes at least one reagent reservoir in fluid communication with the reagent dispensing manifold and the at least one dispensing port and at least on fluidic pump that is configured to pump the reagent from the reagent reservoir to and through the manifold to the at least one dispensing port. The fluidic pump can include a selector valve that is operable to control selected reagents from the reagent reservoir for pumping through the manifold to the at least one dispensing port. The fluidic pump can include a vacuum drive or a pressure or syringe pump to facilitate transfer of the reagents from the reagent reservoir to be dispensed by the dispensing port.

In some embodiments, the device further includes a heating unit configured to heat the plurality of microfluidic chips and an imaging module to image the microfluidic channel of a respective microfluidic chip. The imaging module can be arranged on the platform in an imaging area separate from a sequencing area where the plurality of microfluidic chips are arranged for receiving reagents and sequencing. A robotic arm can transfer the microfluidic chips from the sequencing area to the imaging area. The robotic arm can include a vacuum suction device to secure the microfluidic chips for transfer.

### BRIEF DESCRIPTION OF DRAWINGS

FIG. 1 illustrates the layout of robotic sequencing device with a clustering/sequencing stage, chip storage area and optical imaging/scanning stage.

FIG. 2 illustrates a microfluidic chip with inlet in top glass and outlet in bottom glass.

FIG. 3 illustrates a schematic illustration of a reagent delivery unit.

FIG. 4 illustrates a protype instrument with 3 axis movement, reagent delivery unit, heating stage and waste collecting unit.

FIG. 5 illustrates a reagent/fluidic manifold and dispensing port with Z axis movements.

FIG. 6 illustrates a vacuum drive reagent delivery setup.

FIG. 7 illustrates a pressure drive reagent delivery setup.

### DETAILED DESCRIPTION

Embodiments described herein relate to devices and systems to deliver reagents to microfluidic channels and particularly to nucleotide sequencing devices and systems that can achieve a more efficient use and minimum waste of reagents during fluid exchange to reduce time and cost of nucleotide sequencing. The devices and systems can be useful in, e.g., sequencing for comparative genomics, tracking gene expression, micro RNA sequence analysis, epigenomics, aptamer and phage display library characterization, and other sequencing applications. The devices and systems herein comprise various combinations of optical, mechanical, fluidic, thermal, electrical, and computing devices/aspects. The advantages conferred by the devices and systems described herein include, but are not limited to: (i) reduced device and system manufacturing complexity, operation, and cost, (ii) significantly lower consumable costs (e.g., as compared to those for currently available nucleic acid sequencing systems), (iii) compatibility with typical flow cell surface functionalization methods, (iv) flexible flow control when combined with microfluidic components, e.g., syringe pumps and diaphragm valves, etc., and (v) flexible system throughput.

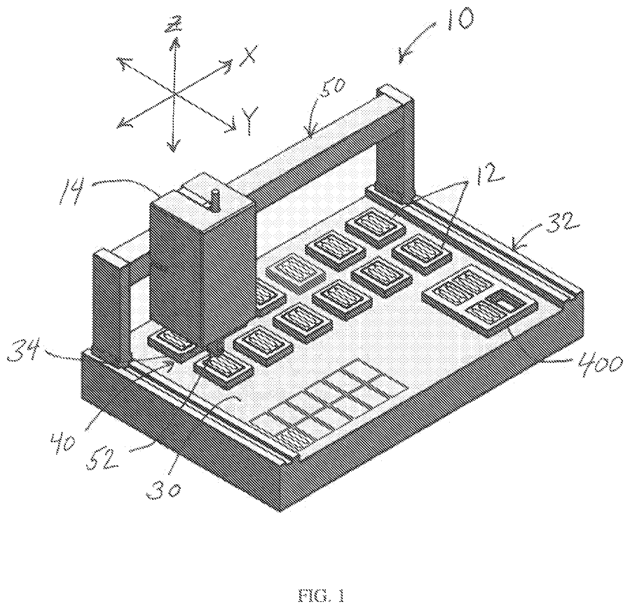

FIG. 1 illustrates a schematic of a robotic nucleotide sequencing device 10 in accordance with an embodiment described herein. The nucleotide sequencing device 10 includes a plurality of microfluidic chips 12 configured for nucleotide sequencing and a reagent delivery unit 14. The microfluidic chips 12 can be arranged on a surface 30 of a platform 32 that extends generally in an x-y plane of an x-y-z coordinate system.

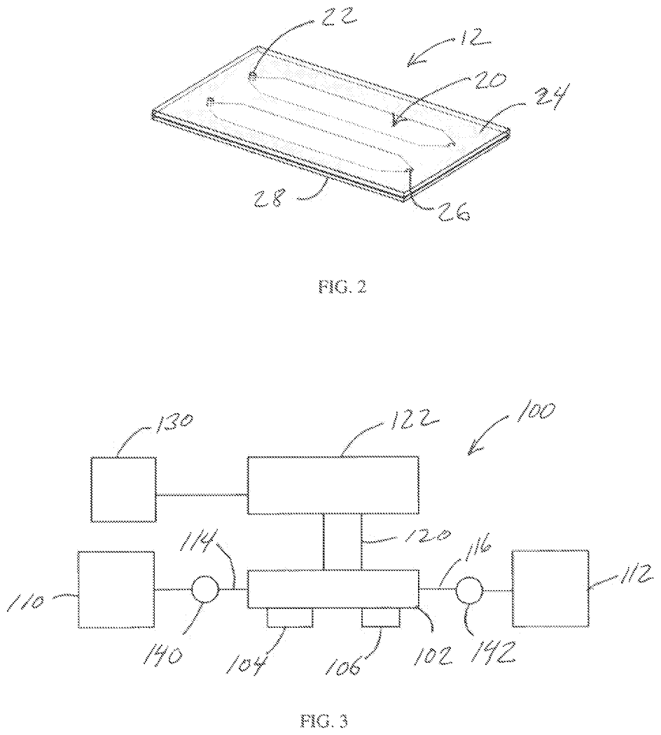

Each microfluidic chip 12 (FIG. 2) includes a microchannel 20 that extends substantially the length of the microfluidic chip 12 from an inlet 22 on an upper surface 24 of the microfluidic chip 12 to an outlet 26 on an opposite lower surface 28 of the microfluidic chip 12. The inlet 22 can be configured to receive a reagent from a reagent dispensing manifold 34 of the reagent delivery unit 14 and the outlet 26 can be in fluid communication with a waste collection unit (not shown).

The platform 30 includes a sequencing area or stage 40 configured to be loaded with more than one microfluidic chip 12 and run different recipes in different channels 20 and chips 12. This design can achieve a very flexible sequencing instrument with flexible throughput from 10 M to 5000M reads on same instruments and the sequencing library can be loaded into channels 20 at any time in any usable channel 20.

The microfluidic chip's 12 inlet 22 can face up toward the reagent dispensing manifold 14 and the outlet 26 can be face down to connect to a waste port of the waste collection unit. This design also enables the microfluidic chip 12 stacking in clustering or other chemistry stepse since the outlet 26 of a first microfluidic chip 12 can be the stacked onto the inlet 22 of a second microfluidic chip 12 which can simplify the microfluidic chip 12 manufacturing process and provide further cost reduction compared to traditional design.

The microchannel 20 can have heights and widths on the order of <1 nm to 1000 μm. For example, in some embodiments a microchannel may have a depth of 1-50 μm, 1-100 μm, 1-150 μm, 1-200 μm, 1-250 μm, 1-300 μm, 50-100 μm, 50-200 μm, or 50-300 μm, or greater than 300 μm, or a range defined by any two of these values. In some embodiments, a microchannel may have a length of less than 0.1 mm, between 0.1 mm and 0.5 mm, between 0.1 mm and 1 mm, between 0.1 mm and 5 mm, between 0.1 mm and 10 mm, between 0.1 mm and 25 mm, between 0.1 mm and 50 mm, between 0.1 mm and 100 mm, between 0.1 mm and 150 mm, between 0.1 mm and 200 mm, between 0.1 mm and 250 mm, between 1 mm and 5 mm, between 1 mm and 10 mm, between 1 mm and 25 mm, between 1 mm and 50 mm, between 1 mm and 100 mm, between 1 mm and 150 mm, between 1 mm and 200 mm, between 1 mm and 250 mm, between 5 mm and 10 mm, between 5 mm and 25 mm, between 5 mm and 50 mm, between 5 mm and 100 mm, between 5 mm and 150 mm, between 5 mm and 200 mm, between 1 mm and 250 mm, or greater than 250 mm, or a range defined by any two of these values. In some embodiments, a microchannel may have a width of less than 0.1 mm, between 0.1 mm and 0.5 mm, between 0.1 mm and 1 mm, between 0.1 mm and 5 mm, between 0.1 mm and 10 mm, between 0.1 mm and 15 mm, between 0.1 mm and 20 mm, between 0.1 mm and 25 mm, between 0.1 mm and 30 mm, between 0.1 mm and 50 mm, or greater than 50 mm, or a range defined by any two of these values. In some embodiments, the microchannel length can be in the micrometer range.

The materials used to fabricate microfluidic chips 12 for the nucleotide sequencing device 10 described herein are often optically transparent to facilitate use with spectroscopic or imaging-based detection techniques. In some instances, the entire microfluidic chip 12 will be optically transparent. In some instances, only a portion of the microfluidic chip 12 (e.g., an optically transparent “window”) will be optically transparent.

The microfluidic chip 12 can be manufactured by a combination of microfabrication processes. The method of manufacturing the microfluidic chip 12 can include providing a surface; and forming at least one channel on the surface. The method of manufacturing can also include providing a first substrate, which has at least a first planar surface, wherein the first surface has a plurality of channels; providing a second substrate having at least a second planar surface; and binding the first planar surface of the first substrate to the second planar surface of the second substrate. In some instances, the channels on the first surface have an open top side and closed bottom side, and the second surface is bond to the first surface through the bottom side of the channels and therefore leaving the open top side of the channels unaffected. In some instances, the method described herein further includes providing a third substrate having a third planar surface, and bonding the third surface to the first surface through the open top side of the channels. The bonding conditions can include, e.g., heating the substrates, or applying an adhesive to one of the planar surfaces of the first or second substrate.

Typically, because the microfluidic chips are microfabricated, substrate materials will be selected based upon their compatibility with known microfabrication techniques, e.g., photolithography, wet chemical etching, laser ablation, laser irradiation, air abrasion techniques, injection molding, embossing, and other techniques. The substrate materials are also generally selected for their compatibility with the full range of conditions to which the microfluidic chips may be exposed, including extremes of pH, temperature, salt concentration, and application of illumination or electric fields. Accordingly, in some preferred aspects, the substrate material may include silica based substrates, such as borosilicate glass, quartz, as well as other substrate materials.

In some embodiments, the manufacturing of the microfluidic chip 12 includes the layering or laminating of two or more layers of substrates, in order to produce the chip 12. For example, in microfluidic chips, the microfluidic elements of the chips are typically produced by laser irradiation, etching or otherwise fabricating features into the surface of a first substrate. A second substrate is then laminated or bonded to the surface of the first to seal these features and provide the fluidic elements of the chip, e.g., the fluid channels.

Referring again to FIG. 1, the reagent dispensing manifold 34 is positioned over the platform 32 and microfluidic chips 12 in a z direction and is configured to move in at least two dimensions or three dimensions, e.g., x, y, and/or z direction, relative to the microfluidic chips 12 by a robotic arm 50. The reagent dispensing manifold 14 can be configured to carry out dispensing of reagents from reagent reservoirs (not shown) through at least one reagent dispensing port 52 to the inlets of the microchips 12 arranged on the platform 30 when certain reagents are requested by chips 12. The at least one dispensing port 52 can form a leak-proof fluid connection with each inlet of the microchannels after movement of the reagent dispensing manifold 34. The at least one dispensing port 52 is configured to be disconnected from the inlet of the microchannel after delivery of a reagent and/or upon movement of the reagent dispensing manifold 34. The reagent delivery unit 14 and reagent dispensing manifold 34 can aspirate quantities of liquid up into, and deposit out those quantities of liquid from, at least one dispensing port 52. The motions and operation of the reagent delivery unit 14 and reagent dispensing manifold 34 is typically controlled by a processor (not shown) such that reagent dispensing operations can be automated.

Advantageously, the reagent delivery unit 14 can be configured so that any pumps, sensors, sample identification verifier, and other items, move with it, and therefore minimize the number of control lines that move across the device 10 or platform 30 during use, and reduces the likelihood that such control lines will become tangled during motion of the reagent delivery unit 14. In some embodiments, reagent delivery unit 14, reagent dispensing manifold 34 are the only items undergoing motion, and remain in communication with other components that are fixed at various points within the device 10. The reagent delivery unit 14 can also be configured to align the at least one dispensing port 52, with the microfluidic chip inlets 22, using a motorized alignment plate.

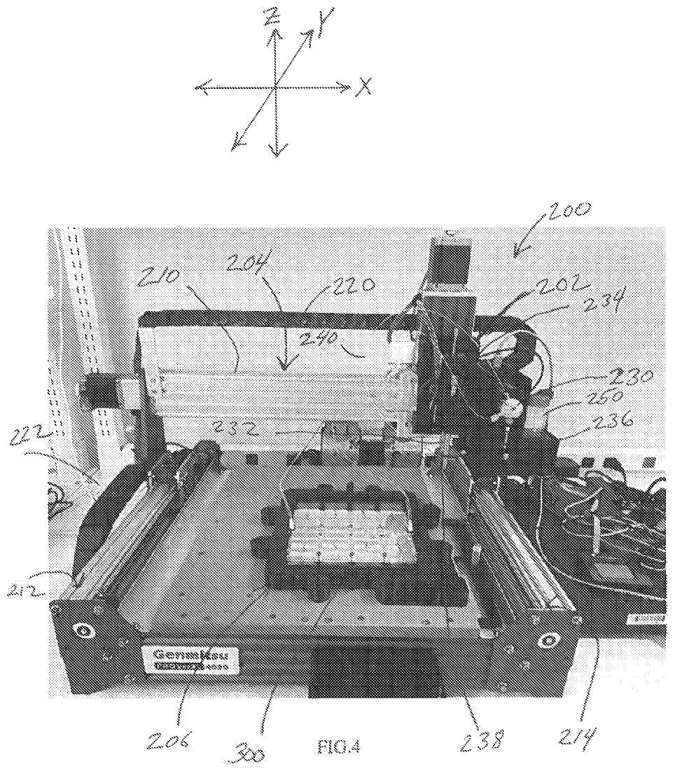

FIG. 3 shows, schematically, components of the reagent delivery unit 100 as further described herein. The layout of the components in FIG. 3 is for convenience only, and one of skill in the art would appreciate that other arrangements are possible, depending upon environment and other factors. The reagent delivery unit 100 includes a reagent delivery manifold 102 that has two dispensing ports 104 and 106 mounted to it. Other numbers of dispensing ports, such as 1, 3, 4, 5, 6, 78, 9, and 10, are consistent therewith. The dispensing ports 104 and 106 are fluidly connected to respective reagent reservoirs 110 and 112 via separate connection lines 114 and 116 such that individual or separate reagents pass from the reagent reservoirs 110 and 112 through respective connection lines 114 and 116 and reagent dispensing manifold 102 and directly to the respective dispensing ports 104 and 106 without traveling through a common line. The reagent dispensing manifold 102 is movably attached via a connecting member 120 to a mount 122 of the reagent delivery unit 100. The relative position of the reagent dispensing manifold 102 and the mount 122, in the z-direction as shown, can be controlled by Z-motor 130, which is electrically coupled via an electrical connection to the connecting member 120 and the mount 122. In some embodiment, the Z-motor 130 can receive instructions from a processor (not shown) via an electrical connection. In other embodiments, the Z-motor 130 can control the relative position of reagent dispensing manifold 102 and mount 122 by moving reagent dispensing manifold 102. In still other embodiments, the Z-motor 130 is coupled to mount 122 and achieves similar relative motion of mount 122 and support 102. Such relative motion can be accomplished by any suitable mechanical movement device, such as gearing, or a rack and pinion assembly, or a lead screw, the details of which are not shown in FIG. 3.

Optionally, the reagent delivery unit 100 can include a sensor (not shown) configured to sense when vertical motion of the reagent dispensing manifold 102 or mount 122 is obstructed, and to provide a suitable signal, e.g., via an electrical connection (not shown), directly to a processor (not shown), or indirectly (not shown) via printed circuit board (not shown). Thus sensor can be mounted on the reagent dispensing manifold 102 or on the mount 122, depending on matters of design choice.

Valves 140 and 142 are associated with each connection line 114 and 116 and dispensing port 104 and 106, and serve to control operation of each dispense port 104 and 106 such as by, for example, controlling when to reduce pressure, thereby causing a suction operation, or to increase pressure, thereby causing a dispense operation. Each valve 140 and 142 is connected to (including being in fluid communication with) reagent dispensing manifold 102.

Operation of the reagent delivery unit 100 is typically controlled by a printed circuit board (PCB) (not shown) to which it is connected via an electrical connection. Thus, the suction and dispense operations can be precisely controlled, by signals from the PCB, so that accurate volumetric control is achieved. In some embodiments, calibration of the reagent delivery unit 100 is required so that the amount of time to force or to suction air that is required to dispense or aspirate a desired volume of reagents is known. Thus, the time between, e.g., a valve opening and valve closing, as controlled by signals, is known and can be incorporated into the control software.

FIG. 4 is an image illustrating an example of a nucleotide sequencing device 200 including the reagent delivery unit 202, as described herein. It would be understood by one of ordinary skill in the art that such components, their relative configuration, number, and orientation, are exemplary, and that the degrees of freedom of motion, and accuracy of positioning and dispensing, consistent with the description herein may be achieved by other such configurations. For example, where one or more mounts are shown, other embodiments may have different numbers of mounts.

The nucleotide sequencing device 200 includes a gantry 204 that provides movement of an attached reagent delivery unit 202 relative to a platform 206 on which is arranged a plurality of microchips 208. The gantry 204 includes a horizontal rail 210 to permit movement of the reagent delivery unit 202 in the x-direction, controlled by the controller assembly. Orthogonally disposed rails 212 and 214 permit movement of the attached reagent delivery unit 202 in the y-direction of the rails 212 and 214.

Control belts 220 and 222 are disposed orthogonal to one another, and provide movement of the reagent delivery 202 in two orthogonal directions, generally in an x-y plane of an x-y-z coordinate system along the horizontal rail 210 and orthogonally disposed rails 212 and 214. The control belts 220 and 222 can further hold include electrical cables, and are disposed to permit motion in a horizontal plane. The control belts 220 and 222 permit easy motion of the reagent delivery 202 without entangling various electrical cables. The electrical cables can supply control signals to a control assembly, which houses electrical circuitry to control operation of the reagent delivery unit 202 and a pump/valve 230 of the reagent delivery unit 202. The reagent delivery unit is thereby capable of moving in two horizontal directions (x-y axis).

A vertically movable extending shaft 232 of the reagent delivery unit 202 coupled to a mount 234 provides movement of a reagent dispensing manifold 236 and reagent dispensing port 238 of the reagent delivery unit 202 in the Z-direction. An electrical cable can supply control signals to the reagent delivery unit 202 which is coupled to a motor for accomplishing vertical motion, and thereby permits such motion to be controlled.

The gantry 204 and shaft 232 thus permit, overall, three degrees of translational freedom of the reagent dispensing manifold 236 and dispensing port 238. Further embodiments, not herein described, can comprise a gantry having fewer than three degrees of translational freedom. The gantry thus provides two axes of belt-driven slides actuated by encoded stepper motors. The gantry slides can be mounted on a framework of structural angle aluminum or other equivalent material, particularly a metal or metal alloy. Slides aligned in x-and y-directions facilitate motion of the dispenser across an array of microfluidic chips, and in a direction along a given holder, respectively. The z-axis of the gantry can be associated with a variable force sensor which can be configured to control the extent of vertical motion of the shaft, mount, and dispenser port during reagent dispensing operations.

The translational motions in three dimensions of the reagent delivery unit 202, reagent dispensing manifold 236, and dispensing ports 236 can be controlled by a microprocessor (not shown). Each dispensing port 238 includes a separate connection line 240 to a respective reagent reservoir 250. This design enables simplification of assembly of the nucleotide sequencing device 200, minimizes contamination of reagents and cross-contamination of samples between different instances of operation of the device 200, increases efficiency of pumping (minimal dead volume) and enables easy maintenance and repair of the device 200. This arrangement also enables easy upgrading of features in the reagent delivery unit 202 and reagent dispensing manifold 236, such as dispensing ports and connection lines to different reagent reservoirs as well as individual and independent pump control for each reagent dispensing port.

It will be appreciated that reagent delivery unit 202 can be configured to carry out fluid transfer operations on two or more dispensing ports simultaneously, such as when operating under instructions received from one or more electrical controllers.

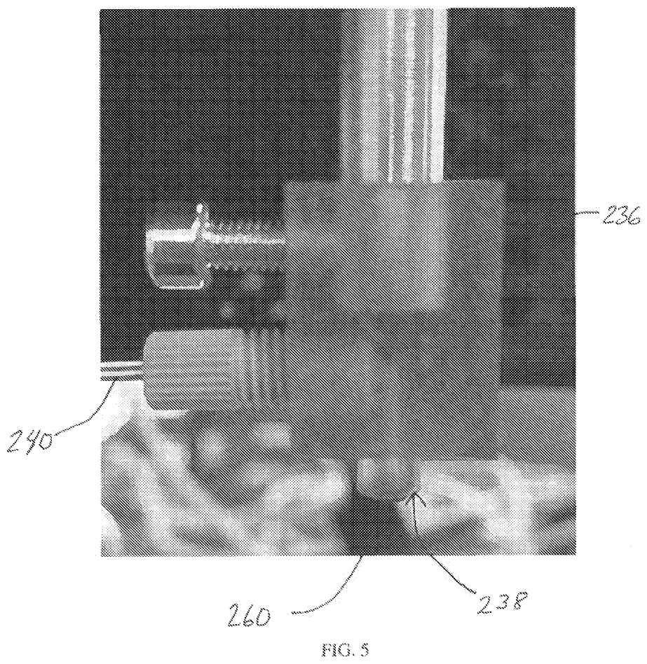

FIG. 5 is an image of a reagent dispensing manifold 236 of the reagent delivery unit 202 of FIG. 4 with Z-axis movement. The reagent dispensing manifold 236 is in fluid communication with connection line 240 that is in fluid communication with a pump and/or valve 230 and a reagent reservoir 250. The dispensing port 238 is mounted to the reagent dispensing manifold 236, which is attached to the shaft 232 of the reagent delivery unit and permits the reagent dispensing port 236 to move up and down vertically. The manner of mounting can be via a mechanical fastener, such as one or more screws.

The reagent dispensing port 238 includes an O-ring 260 that can form a leak-proof fluid connection with inlets of the microchannels (not shown) after movement the reagent dispensing manifold 236 and/or dispensing port 238. Any expensive reagents e.g., incorporation mixture, cleavage mixture, or enzyme mixture will have their own dispensing ports which eliminates the needs to wash off shared common volume.

In most conventional nucleotide sequencing device designs that include a common line from different reagent reservoirs to a single reagent dispensing port, the common line can have a length from about 30 mm to about 300 mm with inner diameter ranged from about 0.5 mm to about 2 mm. The volume of the common line can range from 6 μL to 1000 μL. The common volume consumes large part of reagents that used for sequencing protocol especially for small flow chip format. A 31×3.2×0.08 mm microfluidic channel only needs about 8 μL of reagents to fill the whole channel which is less than the common line volume which is about 20 μL if the internal diameter is 0.5 mm and length is 100 mm. The common line volume limits the minimum reagent consumption of small format sequencing instrument and can block the further price reduction of low throughput sequencing instrument.

In contrast, for the nucleotide sequencing device described herein the minimum reagent consumption is proportional to the dimension of microfluidic channel not common line by assigning all the expensive reagents to dedicated reagent dispensing ports. In addition, the reagent that is injected into the microfluidic channels can be withdrawn to the reagent reservoir by aspirating or suction of the reagent from the channel back to dispensing port since there is no cross contamination between reagents which is common in traditional designs employing a common line.

In some embodiments, the reagent dispensing port 238 can be configured such that a force acting upwardly against the port 238, such as created when the O-ring 260 of the dispensing port 238 meets the inlet of a microfluidic chip, can be sensed through a relative motion between the reagent dispensing port 238 and a force sensor (not shown). The force sensor can be in communication with a processor or controller on the PC board that controls at least the vertical motion of the dispensing port 238 so that the processor or controller can send instructions to arrest the vertical motion of the dispenser port 238 upon receiving an appropriate signal from the force sensor.

The reagent delivery unit 202 can be configured to dispense reagent into a microfluidic chip. Typically, the reagent delivery unit 202 is configured to accept or dispense, in a single operation, an amount of about 10 μl of reagent or less, such as an amount of fluid in the range of about 0.1 μl to about 10 μl.

The nucleotide sequencing device described herein can provide fluid flow control capability for delivering samples or reagents to the one or more microchannels of microfluidic chips connected to the dispensing ports. Reagents and buffers may be stored in bottles, reagent and buffer cartridges, or other suitable containers that are connected to the reagent dispensing manifold by means of tubing and valve manifolds. The device may also include processed sample and waste reservoirs in the form of bottles, cartridges, or other suitable containers for collecting fluids downstream of the microfluidic chips. In some embodiments, a fluid flow control module may provide programmable switching of flow between different sources, e.g., sample or reagent reservoirs or bottles located in the device and different dispensing ports to the microchannels of the microfluidic chip. In some embodiments, the fluid flow control module may provide programmable switching of flow between the dispensing ports sample reservoirs, waste reservoirs, etc., connected to the system. In some instances, samples, reagents, and/or buffers may be stored within reservoirs that are integrated into the reagent dispensing manifold itself.

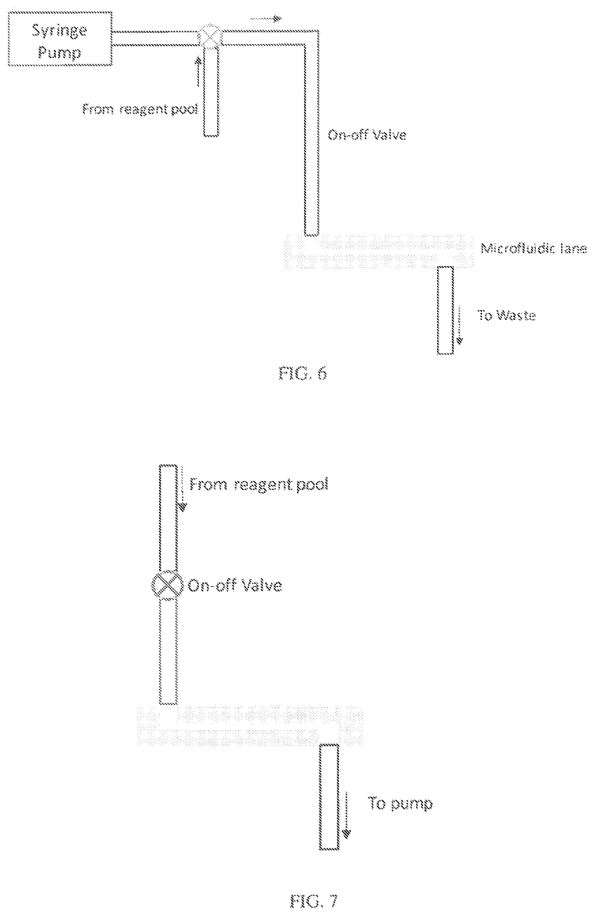

Control of fluid flow through the reagent dispensing manifold, reagent dispensing ports, and microchannels of microfluidic chips will typically be performed through the use of pumps (or other fluid actuation mechanisms) and valves (e.g., programmable pumps and valves). Examples of pumps include, but are not limited to, syringe pumps, programmable syringe pumps, peristaltic pumps, diaphragm pumps, and the like. Examples of valves include, but are not limited to, check valves, electromechanical two-way or three-way valves, pneumatic two-way and three-way valves, and the like. In some embodiments, fluid flow through the reagent dispensing manifold, reagent dispensing ports, and microchannels of microfluidic chips may be controlled by means of applying positive pneumatic pressure to one or more inlets of the reagent and buffer containers, or to inlets incorporated into reagent dispensing manifold or by means of drawing a vacuum at one or more outlets of waste reservoir(s), or at one or more outlets incorporated into microchannels of the microfluidic chips. For example, as illustrated in FIG. 6, in the pressure drive mode, the selected reagent is aspirated from reagent pool via selector valve integrated with pump and injected to the microfluidic channel via the pump's selector valve. The outlet of microfluidic lane can be open to atmosphere or connected to vacuum source in this case. As illustrated in FIG. 7 in the vacuum drive mode, the outlet of microfluidic channel is connected to vacuum source such as syringe pump or vacuum generator. The upstream of microfluidic channel is controlled by valve which will be turned on during reagent delivery.

In some instances, different modes of fluid flow control are utilized at different points in an assay or analysis procedure, e.g., forward flow (relative to the inlet and outlet for a given microchannel of a microfluidic chip), reverse flow, oscillating or pulsatile flow, or combinations thereof. In some applications, oscillating or pulsatile flow may be applied, for example, during assay wash/rinse steps to facilitate complete and efficient exchange of fluids within the one or more microchannels of a microfluidic chip.

In some cases, different fluid flow rates may be utilized at different points in the assay or analysis process workflow, for example, in some instances, the volumetric flow rate may vary from −100 μl/sec to +100 μl/sec. In some embodiment, the absolute value of the volumetric flow rate may be at least 0.001 μl/sec, at least 0.01 μl/sec, at least 0.1 μl/sec, at least 1 μl/sec, at least 10 μl/sec, or at least 100 μl/sec. In some embodiments, the absolute value of the volumetric flow rate may be at most 100 μl/sec, at most 10 μl/sec, at most 1 μl/sec, at most 0.1 μl/sec, at most 0.01 μl/sec, or at most 0.001 μl/sec. The volumetric flow rate at a given point in time may have any value within this range, e.g., a forward flow rate of 2.5 μl/sec, a reverse flow rate of −0.05 μl/sec, or a value of 0 ml/sec (i.e., stopped flow).

Referring again to FIG. 4, the nucleotide sequencing device 200 can include temperature control functionality for the purpose of facilitating the accuracy and reproducibility of assay or analysis results. Examples of temperature control components that may be incorporated into the device 200 for controlling the temperature of individual or respective microfluidic chips 12 include resistive heating elements, infrared light sources, Peltier heating or cooling devices, heat sinks, thermistors, thermocouples, and the like. In some embodiments, a temperature control module or heating stage 300 (or “temperature controller”) may provide for a programmable temperature change at a specified, adjustable time prior to performing specific assay or analysis steps. In some instances, the temperature controller may provide for programmable changes in temperature over specified time intervals. In some embodiments, the temperature controller may further provide for cycling of temperatures between two or more set temperatures with specified frequency and ramp rates so that thermal cycling for amplification reactions may be performed.

In some embodiments, the nucleotide sequencing device 10 can further include imaging capabilities 400, such as optical imaging or other spectroscopic measurement capabilities, for imaging the microchannel of the microfluidic chip. As illustrated in FIG. 1, the imaging capabilities can be separated on the device 10 from areas 40 where the nucleic sequencing occur, e.g., heating/sequencing chemistry stage. Microfluidic chips 12 selected for imaging can be moved by a robotic arm via vacuum suction cup (not shown) and relocated to the designed image stage 400 of the platform 30. The microfluidic chip 12 can be naturally cooled to room temperature once removed from a heat source at heating/sequencing chemistry stage and with the help of heat sink at the imaging stage 400.

The imaging capability can include any of a variety of imaging modes known to those of skill in the art including bright-field, dark-field, fluorescence, luminescence, or phosphorescence imaging. In some embodiments, the microfluidic chip comprises a window that allows at least a part of the microchannel to be illuminated and imaged.

In some embodiments, single wavelength excitation and emission fluorescence imaging may be performed. In other embodiments, dual wavelength excitation and emission (or multi-wavelength excitation or emission) fluorescence imaging may be performed. In some instances, the imaging module is configured to acquire video images. The choice of imaging mode may impact the design of the microfluidic chips in that all or a portion of the microfluidic chips will necessarily need to be optically transparent over the spectral range of interest. In some embodiments, a series of images may be “tiled” to create a single high resolution image of the microchannel within the microfluidic chip.

A spectroscopy or imaging module may comprise, e.g., a microscope equipped with a CMOS of CCD camera. In some instances, the spectroscopy or imaging module may comprise, e.g., a custom instrument configured to perform a specific spectroscopic or imaging technique of interest. In general, the hardware associated with the imaging module may include light sources, detectors, and other optical components, as well as processors or computers.

Any of a variety of light sources may be used to provide the imaging or excitation light, including but not limited to, tungsten lamps, tungsten-halogen lamps, arc lamps, lasers, light emitting diodes (LEDs), or laser diodes. In some instances, a combination of one or more light sources, and additional optical components, e.g. lenses, filters, apertures, diaphragms, mirrors, and the like, may be configured as an illumination system (or sub-system).

Any of a variety of image sensors may be used for imaging purposes, including but not limited to, photodiode arrays, charge-coupled device (CCD) cameras, or complementary metal-oxide-semiconductor (CMOS) image sensors. As used herein, “imaging sensors” may be one-dimensional (linear) or two-dimensional array sensors. In many instances, a combination of one or more image sensors, and additional optical components, e.g., lenses, filters, apertures, diaphragms, mirrors, and the like, may be configured as an imaging system (or sub-system). In some instances, e.g., where spectroscopic measurements are performed by the system rather than imaging, suitable detectors may include, but are not limited to, photodiodes, avalanche photodiodes, and photomultipliers.

The hardware components of the spectroscopic measurement or imaging module may also include a variety of optical components for steering, shaping, filtering, or focusing light beams through the system. Examples of suitable optical components include, but are not limited to, lenses, mirrors, prisms, apertures, diffraction gratings, colored glass filters, long-pass filters, short-pass filters, bandpass filters, narrowband interference filters, broadband interference filters, dichroic reflectors, optical fibers, optical waveguides, and the like. In some instances, the spectroscopic measurement or imaging module may further comprise one or more translation stages or other motion control mechanisms for the purpose of moving capillary flow cell devices and cartridges relative to the illumination and/or detection/imaging sub-systems, or vice versa.

In some embodiments, the nucleotide sequencing device may further comprise a computer (or processor) and computer-readable medium that includes code for providing image processing and analysis capability. Examples of image processing and analysis capability that may be provided by the software include, but are not limited to, manual, semi-automated, or fully-automated image exposure adjustment (e.g., white balance, contrast adjustment, signal-averaging and other noise reduction capability, etc.), automated edge detection and object identification (e.g., for identifying clonally-amplified clusters of fluorescently-labeled oligonucleotides on the surfaces of microchannels), automated statistical analysis (e.g., for determining the number of clonally-amplified clusters of oligonucleotides identified per unit area on the surfaces of microchannels, or for automated nucleotide base-calling in nucleic acid sequencing applications), and manual measurement capabilities (e.g., for measuring distances between clusters or other objects, etc.). Optionally, instrument control and image processing/analysis software may be written as separate software modules. In some embodiments, instrument control and image processing/analysis software may be incorporated into an integrated package.

In some instances, the device may comprise a computer (or processor) and a computer-readable medium that includes code for providing a user interface as well as manual, semi-automated, or fully-automated control of all system functions, e.g., control of the fluidics module, the temperature control module, and/or the spectroscopy or imaging module, as well as other data analysis and display options. The system computer or processor may be an integrated component of the device (e.g., a microprocessor or mother board embedded within the device) or may be a stand-alone module, for example, a main frame computer, a personal computer, or a laptop computer that is part of a system that includes the device. Examples of fluid control functions provided by the system control software include, but are not limited to, volumetric fluid flow rates, fluid flow velocities, the timing and duration for sample and reagent addition, buffer addition, and rinse steps. Examples of temperature control functions provided by the system control software include, but are not limited to, specifying temperature set point(s) and control of the timing, duration, and ramp rates for temperature changes. Examples of spectroscopic measurement or imaging control functions provided by the system control software include, but are not limited to, autofocus capability, control of illumination or excitation light exposure times and intensities, control of image acquisition rate, exposure time, and data storage options.

In some embodiments, the disclosed systems may comprise one or more processors or computers. The processor may be a hardware processor such as a central processing unit (CPU), a graphic processing unit (GPU), a general-purpose processing unit, or a computing platform. The processor may be comprised of any of a variety of suitable integrated circuits, microprocessors, logic devices, field-programmable gate arrays (FPGAs) and the like. In some instances, the processor may be a single core or multi core processor, or a plurality of processors may be configured for parallel processing. Although the disclosure is described with reference to a processor, other types of integrated circuits and logic devices are also applicable. The processor may have any suitable data operation capability. For example, the processor may perform 512 bit, 256 bit, 128 bit, 64 bit, 32 bit, or 16 bit data operations.

The processor or CPU can execute a sequence of machine-readable instructions, which can be embodied in a program or software. The instructions may be stored in a memory location. The instructions can be directed to the CPU, which can subsequently program or otherwise configure the CPU to implement, e.g., the system control methods of the present disclosure. Examples of operations performed by the CPU can include fetch, decode, execute, and write back.

Some processors are a processing unit of a computer system. The computer system may enable cloud-based data storage and/or computing. In some instances, the computer system may be operatively coupled to a computer network (“network”) with the aid of a communication interface. The network may be the internet, an intranet and/or extranet, an intranet and/or extranet that is in communication with the internet, or a local area network (LAN). The network in some cases is a telecommunication and/or data network. The network may include one or more computer servers, which may enable distributed computing, such as cloud-based computing.

The computer system may also include computer memory or memory locations (e.g., random-access memory, read-only memory, flash memory), electronic storage units (e.g., hard disk), communication interfaces (e.g., network adapters) for communicating with one or more other systems, and peripheral devices, such as cache, other memory units, data storage units and/or electronic display adapters. In some instances, the communication interface may allow the computer to be in communication with one or more additional devices. The computer may be able to receive input data from the coupled devices for analysis. Memory units, storage units, communication interfaces, and peripheral devices may be in communication with the processor or CPU through a communication bus (solid lines), such as may be incorporated into a motherboard. A memory or storage unit may be a data storage unit (or data repository) for storing data. The memory or storage units may store files, such as drivers, libraries and saved programs. The memory or storage units may store user data, e.g., user preferences and user programs.

The system control, image processing, and/or data analysis methods as described herein can be implemented by way of machine-executable code stored in an electronic storage location of the computer system, such as, for example, in the memory or electronic storage unit. The machine-executable or machine-readable code can be provided in the form of software. During use, the code can be executed by the processor. In some cases, the code can be retrieved from the storage unit and stored in memory for ready access by the processor. In some situations, the electronic storage unit can be precluded, and machine-executable instructions are stored in memory.

In some instances, the code may be pre-compiled and configured for use with a machine having a processer adapted to execute the code. In some instances, the code may be compiled during runtime. The code can be supplied in a programming language that can be selected to enable the code to execute in a pre-compiled or as-compiled fashion.

Some aspects of the device, system and methods provided herein can be embodied in software. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

In some instances, the system control, image processing, and/or data analysis methods of the present disclosure may be implemented by way of one or more algorithms. An algorithm may be implemented by way of software upon execution by the central processing unit.

In some embodiments, the devices and systems described herein can be used for nucleic acid sequencing of samples. Nucleic acid sequencing provides one non-limiting example of an application for the disclosed device. Many “second generation” and “third generation” sequencing technologies utilize a massively parallel, cyclic array approach to sequencing-by-synthesis (SBS), in which accurate decoding of a single-stranded template oligonucleotide sequence tethered to a solid support relies on successfully classifying signals that arise from the stepwise addition of A, G, C, and T nucleotides by a polymerase to a complementary oligonucleotide strand. These methods typically require the oligonucleotide template to be modified with a known adapter sequence of fixed length, affixed to a solid support (e.g., the microchannel surface(s) of the disclosed microfluidic chip) in a random or patterned array by hybridization to surface-tethered probes of known sequence that is complementary to that of the adapter sequence, and then probed through a cyclic series of single base addition primer extension reactions that use, e.g., fluorescently-labeled nucleotides to identify the sequence of bases in the template oligonucleotides. These processes thus require the use of miniaturized fluidics systems that offer precise, reproducible control of the timing of reagent introduction to the flow cell in which the sequencing reactions are performed, and small volumes to minimize the consumption of costly reagents.

The devices and systems described herein can be used in a variety of applications such as sequencing analysis to improve the efficient use of the costly reagents. For examples, a method of sequencing a nucleic acid sample and a second nucleic acid sample can include delivering a plurality of oligonucleotides to a microchannel of an at least partially transparent microfluidic chip; delivering a first nucleic acid sample to the microchannel; delivering a plurality of nonspecific reagents through a first reagent dispensing port to the microchannel; delivering a specific reagent through a second dispensing port different than the first dispensing port to the microchannel; and visualizing a sequencing reaction in the microchannel. In some aspects, the described method can include selecting the plurality of oligonucleotides to sequence a eukaryotic genome. In some aspects, the described method can include selecting the plurality of oligonucleotides to sequence a prokaryotic genome. In some aspects, the described method can include selecting the plurality of oligonucleotides to sequence a transcriptome.

The described devices and systems can also be used in a method of reducing a reagent used in a sequencing reaction, comprising providing a first reagent in a first reservoir; providing a second reagent in a first second reservoir, wherein each of the first reagent of the first reservoir and the second reagent of the second reservoir are sequentially dispensed or introduced to a microchannel of a microfluidic chip through separate connection lines and reagent dispensing ports that are not continuously fluidly connected to the microchannel of the microfluidic chip.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A nucleotide sequencing device comprising:
a plurality microfluidic chips configured for nucleic acid sequencing wherein each microfluidic chip includes a microchannel having an inlet configured to receive a reagent and an outlet in fluid communication with a waste collection unit; and
a reagent dispensing manifold including at least one reagent dispensing port, the reagent dispensing manifold being operable to move in at least two dimensions relative to the plurality of microfluidic chips to allow the at least one dispensing port to form a leak-proof fluid connection with each inlet of the microchannels after movement, wherein the at least one dispensing port is configured to be disconnected from the inlet of the microchannel after delivery of a reagent and/or upon movement of the reagent dispensing manifold.

2. The device of claim 1, further comprising a platform that extends generally in an x-y plane of an x-y-z coordinate system, wherein the plurality of microfluidic chips are arranged on a surface of the platform and the reagent dispensing manifold is positioned over the platform in a z direction.

3. The device of claim 2, further comprising a robotic arm that moves the reagent dispensing manifold in x, y, and/or z direction to position the dispensing port over the inlet of a respective microfluidic chip.

4. The device of claim 1, further comprising at least one reagent reservoir in fluid communication with the reagent dispensing manifold and the at least one dispensing port.

5. The device of claim 4, further comprising at least on fluidic pump that is configured to pump the reagent from the reagent reservoir through the manifold to the at least one dispensing port.

6. The device of claim 5, wherein the fluidic pump includes a selector valve, the selector being operable to control select reagents from the reagent reservoir for pumping through the manifold to the at least one dispensing port.

7. The device of claim 5, wherein the fluidic pump includes a vacuum drive.

8. The device of claim 5, wherein the fluidic pump includes a pressure or syringe pump.

9. The device of claim 1, further comprising a heating unit configured to heat the plurality of microfluidic chips.

10. The device of claim 2, further comprising an imaging module to image the microfluic channel of a respective microfluidic chip.

11. The device of claim 1, wherein the imaging module is arranged on the platform in an imaging area separate from a sequencing area where the plurality of microfluidic chips are arranged for receiving reagents.

12. The device of claim 11, including a robotic arm to transfer the microfluidic chips from the sequencing area to the imaging area.

13. The device of claim 12, wherein the robotic arm includes a vacuum suction device secure the microfluidic chips for transfer.

14. The device of claim 1, being free of a common line between the dispensing port and inlet of microfluidic chip.

15. The device of claim 1, wherein the reagent and microchannel being configured for nucleotide sequencing.

16. A method of sequencing nucleic samples, the method comprising:
providing the nucleotide sequencing device according to claim 1;
delivering via the reagent dispensing manifold of the nucleotide sequencing device a plurality of oligonucleotides to a microchannel of an at least partially transparent microfluidic chip;
delivering via the reagent dispensing manifold of the nucleotide sequencing device a first nucleic acid sample to the microchannel;
delivering via the reagent dispensing manifold of the nucleotide sequencing device a plurality of nonspecific reagents through a first reagent dispensing port to the microchannel;
delivering via the reagent dispensing manifold of the nucleotide sequencing device a specific reagent through a second dispensing port different than
the first dispensing port to the microchannel; and
visualizing a sequencing reaction in the microchannel.

17. The method of claim 16, further comprising selecting the plurality of oligonucleotides to sequence a eukaryotic genome or a prokaryotic genome.

18. A method of reducing a reagent used in a sequencing reaction, the method comprising:
providing the nucleotide sequencing device according to claim 1;
providing a first reagent in a first reservoir;
providing a second reagent in a first second reservoir, wherein each of the first reagent of the first reservoir and the second reagent of the second reservoir are sequentially dispensed or introduced to a microchannel of a microfluidic chip through separate connection lines and reagent dispensing ports of the nucleotide sequencing device that are not continuously fluidly connected to the microchannel of the microfluidic chip.
